(19) United States
(12) Patent Application Publication
(10) Pub. No.: US 2005/0177040 A1
(43) Pub. Date: Aug. 11, 2005

(54) System and method for an iterative technique to determine fisher discriminant using heterogenous kernels

(76) Inventors: Glenn Fung, Bryn Mawr, PA (US); Murat Dundar, Malvern, PA (US); Jinbo Bi, Exton, PA (US); R. Bharat Rao, Berwyn, PA (US)

Correspondence Address: Siemens Corporation;Intellectual Property Department, 170 Wood Avenue South, Iselin, NJ, 08830, US

(21) Appl. No.: 11/050,599

(22) Filed: Feb. 3, 2005

Related U.S. Application Data
(60) Provisional application No. 60/542,416, filed on Feb. 6, 2004.

(51) Int. Cl. A61B 5/05

(52) U.S. Cl. 600/407

(57) ABSTRACT

A method and device with instructions for analyzing an image data-space includes creating a library of one or more kernels, wherein each kernel from the library of the kernels maps the image data-space to a first data-space using at least one mapping function; and learning a linear combination of kernels in an automatic manner to generate at least one of a classifier and a regressor which is applied to the first data-space. The linear combination of kernels is used to generate a classified image-data space to detect at least one of the candidates in the classified image-data space.

### CROSS-REFERENCE TO RELATED APPLICATION

This application claims the benefit of U.S. Provisional Application No. 60/542,416 filed on Feb. 6, 2004, titled as “A Fast Iterative Algorithm for Fisher Discriminant Using Heterogeneous Kernels”, entire contents of which are incorporated herein by reference.

### TECHNICAL FIELD

The present invention generally relates to medical imaging and more particularly to applying mathematical techniques for detecting candidate anatomical abnormalities as shown in medical images.

### DISCUSSION OF THE RELATED ART

The field of medical imaging has seen significant advances since the time X-Rays were first used to determine anatomical abnormalities. Medical imaging hardware has progressed in the form of newer machines such as Medical Resonance Imaging (MRI) scanners, Computed Axial Tomography (CAT) scanners, etc. Because of large amount of image data generated by such modern medical scanners, there is a need for developing image processing techniques that automatically determine the presence of anatomical abnormalities in scanned medical images.

Recognizing anatomical structures within digitized medical images presents multiple challenges. One concern is related to the accuracy of recognition. Another concern is the speed of recognition. Because medical images are an aid for a doctor to diagnose a disease or condition, the speed of recognition is of utmost important to aid the doctor in reaching an early diagnosis. Hence, there is a need for improving recognition techniques that can provide accurate and fast recognition of anatomical structures in medical images.

Digital medical images are constructed using raw image data obtained from a scanner, for example, a CAT scanner, MRI, etc. Digital medical images are typically either a 2-D image made of pixel elements or a 3-D image made of volume elements (“voxels”). Such 2-D or 3-D images are processed using medical image recognition techniques to determine presence of anatomical structures such as cysts, tumors, polyps, etc.

A typical image scan generates a large amount of image data, and hence it is preferable that an automatic technique should point out anatomical features in the selected regions of an image to a doctor for further diagnosis of any disease or condition. The speed of processing image data to recognize anatomical structures is critical in medical diagnosis and hence there is a need for a faster medical image processing and recognition technique(s).

One conventional approach to candidate recognition in medical images uses standard Kernel Fisher Discriminant (KFD), but it requires the user to predefine a kernel function. Further, improved performance can be obtained from standard KFD but that requires the kernel parameters to be tuned using cross validation.

### SUMMARY

In one aspect of the invention, a method and device having instructions for analyzing an image data-space includes creating a library or a family of one or more kernels, wherein each kernel from the library of kernels maps the image data-space to a first data-space using at least one mapping function; and learning a linear combination of kernels in an automatic manner to generate at least one of a classifier and a regressor. The linear combination of kernels is used to generate a classified image-data space to detect at least one of the candidates in the classified image-data space.

Another aspect of the invention includes a method for finding a regularized network that solves a nonlinear classification problem, the method includes creating a library of kernels; and calculating a linear combination of the kernels to solve a first convex Quadratic Programming (QP) problem using the linear combination, and to solve a second convex QP problem using the solved first QP to obtain at least one of a classifier and a regressor to generate a classified data space by applying at least one of the classifier and a regressor.

### BRIEF DESCRIPTION OF DRAWINGS

Exemplary embodiments of the present invention are described with reference to the accompanying drawings, of which:

FIG. 1 is a flow-chart for an automatic Kernel Fisher Discriminant (A-KFD) kernel selection technique to determine a classifier in at least one exemplary embodiment of the invention;

FIG. 2 shows an exemplary colon having a polyp shown in a graphical interface in an exemplary embodiment of the invention;

FIG. 3 is a flowchart showing the automatic Kernel Fisher Discriminant (A-KFD) technique that can be used to determine anatomical structures and conditions in a medical image; and

FIG. 4 shows an illustrative computer system in an exemplary embodiment of the invention used to implement at least one embodiment of the invention.

### DETAILED DESCRIPTION OF THE EXEMPLARY EMBODIMENTS

The exemplary embodiments of the present invention will be described with reference to the appended drawings.

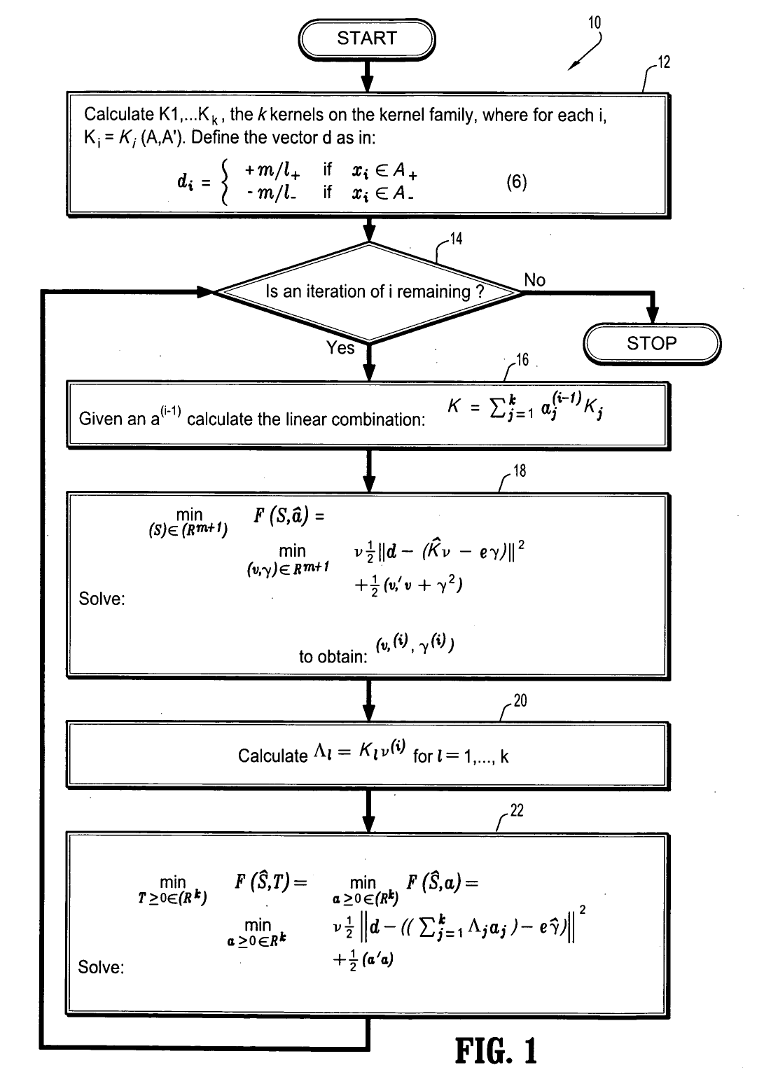

FIG. 1 is a flow-chart for an Automatic Kernel Fisher Discriminant (A-KFD) kernel selection technique in at least one exemplary embodiment of the invention. A relatively fast iterative classification algorithm for KFD uses heterogeneous kernel models. The task of choosing an appropriate kernel is incorporated within the optimization problem to be solved, in contrast with the conventional standard KFD which requires the user to predefine a kernel function. The choice of the kernel can be considered as a linear combination of kernels belonging to a potentially large family of different positive semi-definite kernels.

The complexity of the technique does not increase significantly with respect to the number of kernels in the kernel family. Experiments on several benchmark datasets demonstrate that generalization performance of the technique is not significantly different from that achieved by the conventional standard KFD in which kernel parameters have been tuned using cross validation. Further, as an illustration, a real-life colon cancer dataset can be used in an another exemplary embodiment of the invention to demonstrate the efficiency of the technique.

The goal here is to learn a classifier which can detect regions of abnormalities in an image when a medical expert is viewing it.. A classifier is a function that takes a given vector and maps it to a class label. For instance, a classifier could map a region of colon from a colon CT scan, to a label of “polyp” or “non polyp” (which could be stool, or just the colon wall). The above is an example of a binary classifier, that has just two labels—for illustration purposes, it can be assumed that Class_{—}1 or POSITIVE (is a polyp) or Class_{—}2 or NEGATIVE (is not a polyp), but the same description applies to a classifier that can have many labels (e.g., polyp, stool, colon wall, air, fluid, etc. . . . ), and also it can apply to any data classification problem, medical imaging being an illustration.

A classifier is trained from a training data set, which is a set of samples that have labels (i.e., the label for each sample is known, and in the case of medical imaging the label is typically confirmed either by expert medical opinion or via biopsy truth).

Kernel based methods can be used to solve classification problems. It is known that the use of an appropriate nonlinear kernel mapping is a critical issue when nonlinear hyperplane based methods such as Kernel Fisher Discriminant (KFD) are used for classification. Typically, kernels are chosen by predefining a kernel model (Gaussian, polynomial, etc.) and then followed by adjusting of the kernel parameters by means of a tuning procedure. The kernel selection is based on the classification performance on a subset of the training data that is commonly referred to as the “validation set”. Such manual kernel selection procedure can be computationally very expensive and is particularly prohibitive when the dataset is large; furthermore, there is no certainty that the predefined kernel model is an optimal choice for the classification problem.

A linear combination of kernels formed by a family of different kernel functions and parameters can be used. But still the task of finding an optimal linear combination of the members of the kernel family remains to be completed. Using this approach there is no need to predefine a kernel; instead, a final kernel is constructed according to a specific data classification problem to be solved without sacrificing capacity control. By combining kernels, the hypothesis space is made larger (potentially, but not always), but with appropriate regularization, prediction accuracy is improved which is the ultimate goal for classification.

A linear combination of kernels can lead to considerably more complex optimization problems . Hence, at least one embodiment of the invention uses a fast iterative algorithmic technique that transforms the resulting optimization problem into several relatively computationally less expensive strongly convex optimization problems.

At each iteration, the technique only requires solving of a simple system of linear equations and a relatively small quadratic programming problem with non-negativity constraints, which makes the implementation easier. In contrast with conventional techniques, the complexity of the technique does not depend directly on the number of kernels in the kernel family.

First, the linear classification problem is formulated as a Linear Fisher Discriminant (LFD) problem. Second, it is shown that how the classical Fisher discriminant problem can be reformulated as a convex quadratic optimization problem. Using this equivalent mathematical programming LFD formulation and using mathematical programming duality theory, a Kernel Fisher Discriminant (KFD) is formulated. Third, a formulation is created that incorporates both the KFD problem and the problem of finding an appropriate linear combination of kernels into an quadratic optimization problem with non-negativity constraints on one set of the variables. Fourth, a technique for solving this optimization problem and the complexity and convergence of the technique are discussed. Next, computational results including illustrative ones for a real-life colorectal cancer dataset as well as five other publicly available illustrative datasets are discussed.

The notation used in equations below is discussed next. All vectors will be column vectors unless transposed to a row vector by a prime superscript ′. The scalar (inner) product of two vectors x and y in the n-dimensional real space Rⁿ will be denoted by x′y, the 2-norm of x will be denoted by ∥x∥. The 1 -norm and ∞-norm will be denoted by ∥•∥₁ and ∥•∥_{∞} respectively. For a matrix A ε R^{m×n}, A₁ is the i th row of A which is a row vector in Rⁿ. A column vector of ones of arbitrary dimension will be denoted by e, and the identity matrix of arbitrary order will be denoted by I. For A ε R^{m×n} and \dot{B} ε R^{n×t}, the kernel K(A, B) is an arbitrary function which maps into R^{m×n}×R^{n×l} into R^{n×t}. In particular, if x and y are column vectors in Rⁿ, then K(x′, y) is a real number, K(x′, A′) is a row vector in R^{m} and K(A, A′) is a m×m matrix.

The Linear Fisher's Discriminant (LFD) is discussed next. It is conventionally known that the probability of error due to the Bayes classifier is the best that can be achieved. A major disadvantage of the Bayes error as a criterion, is that a closed form analytical expression is not available for the general case. However, by assuming that classes are normally distributed, standard classifiers using quadratic and linear discriminant functions can be designed.

The Linear Fisher's Discriminant (LFD) arises in the special case when the classes have a common covariance matrix. LFD is a classification method that projects the high dimensional data onto a line (for an exemplary binary classification problem) and performs classification in this one dimensional space. This projection is chosen such that either the ratio of the scatter matrices (between and within classes) or the so called “Rayleigh quotient” is maximized.

More specifically, let A ε R^{m×n} be a matrix containing all the samples and let A_{c} ⊂ A ε R^{l}^{ₑ}^{×n} be a matrix containing the l_{c} labeled samples for class c, xᵢ ε Rⁿ, c ε {±}. Then, the LFD is the projection u, which maximizes,

$$\begin{matrix}J ( α ) = \frac{{u}^{T} {S}_{B} u}{{u}^{T} {S}_{W} u} \\ where & ( 1 ) \\ {S}_{B} = ( {M}_{+} - {M}_{-} ) {( {M}_{+} - {M}_{-} )}^{T} & ( 2 ) \\ {S}_{W} = {\sum }_{c ∈ \{ \pm \}} \frac{1}{{l}_{c}} ( {A}_{c} - {M}_{c} {e}_{{l}_{c}}^{T} ) {( {A}_{c} - {M}_{c} {e}_{{l}_{c}}^{T} )}^{T} & ( 3 )\end{matrix}$$

are the between and within class scatter matrices respectively, and

$${M}_{c} = \frac{1}{{l}_{c}} {A}_{c} {e}_{{l}_{c}} \qquad ( 4 )$$

is the mean of class c and e_{lc} is an l_{c} dimensional vector of ones. Traditionally, the LFD problem has been addressed by solving the generalized eigenvalues problem associated with the Equation (1) above. When classes are normally distributed with equal covariance, a is in the same direction as the discriminant in the corresponding Bayes classifier. Hence, for this special case LFD is equivalent to the Bayes optimal classifier. Although LFD relies heavily on assumptions that are not true in most real world problems, it has proven to be very powerful. Generally, when the distributions are unimodal and separated by the scatter of means, LFD can be a desirable solution. One reason why LFD may be preferred over more complex classifiers is that as a linear classifier it is less prone to overfitting.

For most real world data, a linear discriminant is clearly not complex enough. Classical techniques tackle these problems by using more sophisticated distributions in modeling the optimal Bayes classifier, however these often sacrifice the closed form solution and are computationally more expensive. A relatively new approach in this domain is the kernel version of Fisher's Discriminant. The main ingredient of this approach is the kernel concept, which was originally applied in Support Vector Machines and allows the efficient computation of Fisher's Discriminant in the kernel space. The linear discriminant in the kernel space corresponds to a powerful nonlinear decision function in the input space. Furthermore, different kernels can be used to accommodate the wide range of nonlinearities possible in the data set. A slightly different formulation of the KFD problem based on duality theory which does not require the kernel to be positive semi-definite or what is equivalent, does not need the kernel to comply with Mercer's condition.

Automatic heterogeneous kernel selection for the KFD problem is described next. With the exception of an unimportant scale factor, the LFD problem can be reformulated as the following constrained convex optimization problem:

$${min}_{( u , γ ) ∈ {R}^{m + 1}} v \frac{1}{2} {‖ y ‖}^{2} + \frac{1}{2} ( {u}^{′} u ) \\ s . t . y = d - ( A u - e γ ) . \qquad ( 5 )$$

where m=l₊+l_{·} and d is an m-dimensional vector such that:

$${d}_{i} = \{ \begin{matrix}+ m / {l}_{+} & if {x}_{i} ∈ {A}_{+} \\ - m / {l}_{-} & if {x}_{i} ∈ {A}_{-}\end{matrix} \qquad ( 6 )$$

and the variable v is a positive constant introduced to address the problem of ill-conditioning of the estimated covariance matrices. This constant can also be interpreted as a capacity control parameter. To have strong convexity on all variables of problem (5) an extra term γ² can be introduced on the corresponding objective function. In this case, the regularization term is minimized with respect to both orientation u and relative location to the origin γ. Extensive computational experience indicates that in similar problems this formulation is relatively as good as the classical formulation, with some added advantages such as strong convexity of the objective function. After adding the new term to the objective function of the problem (5) the problem becomes:

$${min}_{( u , γ , y ) ∈ {R}^{m + 1 + m}} v \frac{1}{2} {‖ y ‖}^{2} + \frac{1}{2} ( {u}^{′} u + {γ}^{2} ) \\ s . t . y = d - ( A u - e γ ) . \qquad ( 7 )$$

The Lagrangian of Equation (7) is given by:

$$L ( u , γ , y , υ ) = v \frac{1}{2} {‖ y ‖}^{2} + \frac{1}{2} {‖ [ \begin{matrix}u \\ γ\end{matrix} ] ‖}^{2} - {υ}^{′} ( ( A u - γ e ) + y - d ) \qquad ( 8 )$$

Here v ε R^{m} is the Lagrange multiplier associated with the equality constrained problem (7). Solving for the gradient of (8) equal to zero, we obtain the Karush-Kuhn-Tucker (KKT) necessary and sufficient optimality conditions for our LFD problem with equality constraints as given by:
u−A′v=0
γ+e′v=0
vy−v=0
Au−eγ+γ−d=0 (9)
The first three Equations of (9) give the following expressions for the original problem variables (u, γ, y) in terms of the Lagrange multiplier v:

$$u = {A}^{′} υ , γ = - {e}^{′} υ , y = \frac{υ}{v} . \qquad ( 10 )$$

Replacing these equalities in the last equality of (9) allows obtaining an explicit expression involving v in terms of the problem data A and d, as follows:

$$A {A}^{′} υ + e {e}^{′} υ + \frac{υ}{v} - d = ( H {H}^{′} + \frac{I}{v} ) υ - d = 0 \qquad ( 11 )$$

where H is defined as:
H=[A (−e)]. (12)

From the two first equalities of (10) we have that,

$$[ \begin{matrix}u \\ γ\end{matrix} ] = {H}^{′} υ \qquad ( 13 )$$

Using this equality and pre-multiplying by H′ in (11) we have:

$$( {H}^{′} H + \frac{I}{v} ) [ \begin{matrix}u \\ γ\end{matrix} ] = {H}^{′} d \qquad ( 14 )$$

Solving the linear system of equations (14) gives the explicit solution

$$[ \begin{matrix}u \\ γ\end{matrix} ]$$

to the LFD problem (7). To obtain the “kernelized” version of the LFD classifier equality constrained optimization problem (7) is modified by replacing the primal variable u by its dual equivalent u=A′v from (10) to obtain:

$${min}_{( υ , γ , y ) ∈ {R}^{m + 1 + m}} v \frac{1}{2} {‖ y ‖}^{2} + \frac{1}{2} ( {υ}^{′} υ + {γ}^{2} ) \\ s . t . y = d - ( A {A}^{′} υ - e γ ) . \qquad ( 15 )$$

where the objective function has also been modified to minimize weighted 2-norm sums of the problem variables. If we now replace the linear kernel AA′ by a nonlinear kernel K(A,A′) as defined above, we obtain a formulation that is equivalent to the kernel Fisher discriminant:

$${min}_{( υ , γ , y ) ∈ {R}^{m + 1 + m}} v \frac{1}{2} {‖ y ‖}^{2} + \frac{1}{2} ( {υ}^{′} υ + {γ}^{2} ) \\ s . t . y = d - ( {K ( A , A )}^{′} υ - e γ ) . \qquad ( 16 )$$

Recent (SVM) formulations (d={+1,−1}) with least squares loss are much the same in spirit as the problem of minimizing

$$v \frac{1}{2} {‖ y ‖}^{2} + \frac{1}{2} {w}^{′} w$$

with constraints y=d−(Aw−eγ). Using a similar duality analysis to the one presented before, and then “kernelizing” the objective function is obtained as below:

$$v \frac{1}{2} {‖ y ‖}^{2} + \frac{1}{2} {υ}^{′} {K ( A , A )}^{′} υ . \qquad ( 17 )$$

The regularization term v′K(A A′)v determines that the model complexity is regularized in a reproducing kernel Hilbert space (RKHS) associated with the specific kernel K where the kernel function K has to satisfy Mercer's conditions and K(A,A′) has to be positive semi-definite.

By comparing the objective function (17) to problem (16), it can see that problem (16) does not regularize in terms of RKHS. Instead, the columns in a kernel matrix are simply regarded as new features K(A,A′) of the classification task in addition to original features A. Then, classifiers based on the features introduced by a kernel are constructed in the same way as the build models using original features in A. Further, in a more general framework (regularized networks) our the technique could produce linear classifiers (with respect to the new kernel features K(A,A′) which minimize the cost function regularized in the span space formed by these kernel features. Thus, the requirement for a kernel to be positive semi-definite could be relaxed, at the cost in some cases, of an intuitive geometrical interpretation.

Since a Kernel fisher discriminant formulation is considered here, the kernel matrix will be required to be positive semi-definite. This requirement allows conservation of the geometrical interpretation of the KFD formulation since the kernel matrix can be seen as a “covariance” matrix on the higher dimensional space induced implicitly by the kernel mapping. Next, if instead of the kernel K being defined by a single kernel mapping (i.e., Gaussian, polynomial, etc.), the kernel K is instead composed of a linear combination of kernel functions K_{j,j}=1, . . . k, as below:

$$K ( A , {A}^{′} ) = {\sum }_{j = 1}^{k} {a}_{j} {K}_{j} ( A , {A}^{′} ) , \qquad ( 18 )$$

where aⱼ≧0. The set {K₁(A,A′), . . . , Kₖ(A,A′)} can be seen as a predefined set of initial “guesses” of the kernel matrix. The set {K₁(A,A′), . . . Kₖ(A,A′)} could contain very different kernel matrix models, e.g., linear, Gaussian, polynomial, all with different parameter values. Instead of fine tuning the kernel parameters for a predetermined kernel via cross-validation, the set of values aᵢ≧0 can be optimized in order to obtain a PSD (a matrix A, such that x′Ax>=0 for all x) linear combination

$$K ( A , {A}^{′} ) = {\sum }_{j = 1}^{k} {a}_{j} {K}_{j} ( A , {A}^{′} )$$

suitable for the specific classification problem. Replacing equation (18) in equation (16) and solving for y in and replacing it on the objective function in (16), the KFD problem optimization can be reformulated for heterogeneous linear combinations of kernel as follows:

$${min}_{( υ , γ , a \geq 0 ) ∈ {R}^{m + 1}} v \frac{1}{2} {‖ d - ( ( {\sum }_{j = 1}^{k} {a}_{j} {K}_{j} ) υ - e γ ) ‖}^{2} + \frac{1}{2} ( {υ}^{′} υ ) \qquad ( 19 )$$

where Kⱼ=Kⱼ(A,A′). When considering linear combinations of kernels the hypothesis space may become larger, making the issue of capacity control an important one. If two classifiers have similar training error, a smaller capacity may lead to better generalization on future unseen data. In order to reduce the size of the hypothesis and model space and to gain strong convexity in all variables, an additional regularization term a′a=∥a∥² is added to the objective function of problem (19). The problem then becomes,

$${min}_{( υ , γ , a \geq 0 ) ∈ {R}^{m + 1}} v \frac{1}{2} {‖ d - ( ( {\sum }_{i = 1}^{k} {a}_{i} {K}_{i} ) υ - e γ ) ‖}^{2} + \frac{1}{2} ( {υ}^{′} υ + {γ}^{2} + {a}^{′} a ) \qquad ( 20 )$$

The corresponding nonlinear classifier to this nonlinear separating surface is then:

$$( {\sum }_{j = 1}^{k} ( {a}_{j} {K}_{j} ( {x}^{′} , {A}^{′} ) ) ) υ - γ = \{ \begin{matrix}> 0 , & then x ∈ {A}_{+} , \\ < 0 , & then x ∈ {A}_{-} , \\ = 0 , & then x ∈ {A}_{+} ⋃ {A}_{-} .\end{matrix} \qquad ( 21 )$$

Furthermore, problem (20) can be seen as a biconvex program of the form,

$$\begin{matrix}{min}_{( S , T ) ∈ ( {R}^{m + 1} , {R}^{k} )} F ( S , T ) & ( 22 ) \\ where S = [ \begin{matrix}υ \\ γ\end{matrix} ] and T = a & \end{matrix}$$

When T=\overline{a} is fixed, problem (22) becomes:

$${min}_{( S ) ∈ ( {R}^{m + 1} )} F ( S , \hat{a} ) = {min}_{( υ , γ ) ∈ {R}^{m + 1}} v \frac{1}{2} {‖ d - ( \hat{K} υ - e γ ) ‖}^{2} + \frac{1}{2} ( {υ}^{′} υ + {γ}^{2} ) \qquad ( 23 )$$

where

$$\hat{K} = {\sum }_{j = 1}^{k} {\hat{a}}_{j} {K}_{j} .$$

This is equivalent to solving (16) with K=\hat{K}. On the other hand when

$$\hat{S} = [ \begin{matrix}\hat{υ} \\ \hat{γ}\end{matrix} ]$$

is fixed, problem (22) becomes:

$$\begin{matrix}{min}_{T \geq 0 ∈ ( {R}^{k} )} F ( \hat{S} , T ) = {min}_{a \geq 0 ∈ ( {R}^{k} )} F ( \hat{S} , a ) \\ = {min}_{a \geq 0 ∈ {R}^{k}} v \frac{1}{2} {‖ d - ( ( {\sum }_{j = 1}^{k} {Λ}_{j} {a}_{j} ) - e \hat{γ} ) ‖}^{2} + \frac{1}{2} ( {a}^{′} a )\end{matrix} \qquad ( 24 )$$

where Aⱼ=Kⱼv.

Sub-problem (23) is an unconstrained strongly convex problem for which a unique solution in close form can be obtained by solving a (m+1)×(m+1) system of linear equations. On the other hand, sub-problem (24) is also a strongly convex problems with the simple non-negativity constraint a≧0 on k variables (k is usually very small) for which a unique solution can be obtained by solving a relatively simple quadratic programming problem.

The Automatic kernel selection KFD Algorithm (A-KFD) technique used in an exemplary embodiment shown in a flowchart 10 (hereafter “the Algorithm”) is described next. Given m data points in Rₙ represented by the m×n matrix A and vector L of ±1 labels denoting the class of each row of A, the parameter μ and an initial a⁰ └ [symbol] ^{k}, the nonlinear classifier (21) is generated as follows:

The steps in the flowchart 10 stop when a predefined maximum number of iterations for i is reached or when there is sufficiently little change of the objective function of problem (20) evaluated in successive iterations.

In a step 12, a library of k kernels is generated, where Kⱼ=Kⱼ(A,A′) for each i. Vector d, which represents the given weighted labels as defined in (6),

Iteration testing condition in a step 14 repeats the loop till a predetermined iteration threshold for iterating is reached.

In a step 16, the kernel matrix K is calculated using all kernels on the kernel library using the weight vector aⁱ⁻¹.

In a step 18 a convex quadratic optimization problem is solved to find the separating hyperplane normal vector v⁽ⁱ⁾, based on the kernel automatic defined in the step 16.

In a step 20 calculations necessary to solve the optimization problem of a step 22 are performed. Further, in the step 22, a convex quadratic optimization problem is solved to learn the new weight vector aⁱ that will update the kernel matrix Kin the step 16 of the next iteration.

The kernels are represented by an equation: K(A,A′): R^{m×n}×R^{n×m} [symbol] R^{m×m}. The kernels can be of any type, for example, a Gaussian kernel as represented by equation: K(A,A′)ᵢⱼ=ε^{-μ∥A}^{ᵢ}^{-A}^{ⱼ}^{∥}^{₂}^{²}, i,j=1, . . . , m or a polynomial kernel represented by an equation:. The generated classifier or regressor is represented by the equation:

$${\sum }_{j = 1}^{k} ( {a}_{j} {K}_{j} ( A , {A}^{′} ) ) υ - e γ .$$

The learning process is performed by solving an optimization problem represented by an equation:

$${min}_{( υ , γ , a \geq 0 ) ∈ {R}^{m + 1}} v \frac{1}{2} ‖ d - {( ( {\sum }_{j = 1}^{k} ( {a}_{j} {K}_{j} ( A , {A}^{′} ) ) υ - e γ ) ‖}^{2} + \frac{1}{2} ( {υ}^{′} υ ) + \frac{1}{2} ( {a}^{′} a ) .$$

The process of learning a linear combination involves determining a vector of optimal weights aⁱ⁻¹, in the linear combination of the kernels. Optimal weights are then used to define a kernel K by using the following equation:

$$K = {\sum }_{j = 1}^{k} {a}_{j}^{( i - 1 )} {K}_{j} .$$

The optimal weights define a linear combination of kernels from the kernel library that define an optimal kernel or a kernel very suitable for the classification/regression problem at hand. The vector of weights aⁱ⁻¹ is obtained by solving problem (24).

The optimization problem (20) can be solved using an iteration based on an Alternate Optimization (AO). The AO approach consists in solving a succession of sub-problems that are easier to solve and depend on less variables than the original problem. It is desired that the alternate optimization includes one or more convex problems because convex problems are usually easier to solve and have unique solutions. Further, the optimization problem can be solved using an Expectation Maximization (EM) algorithm where the underlying concept is very similar to AO concept: divide the problem into two sub problems depending only in a subset of the variables and the some iteratively until an optimal solution is obtained.

The process of learning can use at least one of the following techniques: a support vector machines technique, least-square support vector machines technique and a Kernel Fisher Discriminant technique. (i.e., techniques where the classifier to be learnt is a hyperplane that separate the two classes ).

The learning process can also use one or more weak kernels from the library of kernels for automatic feature selection in the image data-space, where the weak kernels depend on only one input feature or attribute. The weak kernels can include weak column kernels that depend on a subset of the centers of the kernels in the library.

As stated above, the final kernel matrix to use in the training process is a linear combination of the kernels in the kernel library where the weights are learned by the algorithm. This can be considered as an implicit automatic kernel selection. In contrast, most kernel-based learning algorithms require expertise and interaction by the user in order to find or design an “appropriate” kernel suitable for the classification problem to solve.

Let Ni be the number of iterations of the algorithm shown in the flowchart 10, when k<<m, which is usually the case, this is when the number of kernels functions considered on the kernel family is much smaller than the number of data points, then the complexity of the algorithm is approximately Nᵢ(O(m³))=O(m³. Since Nᵢ is bounded by the maximum number of iterations and the cost of solving the quadratic programming problem (24), Nᵢ is dominated by the cost of solving the problem (23). In practice, is observed that the Algorithm typically converges in 3-4 iterations (3 or 4) to a local solution of problem (20).

Since each of the two optimization problems ((23) and (24)) that are required to be solved by the A-KFD algorithm are strongly convex and thus each of them have a unique minimizer, the A-KFD algorithm can also be interpreted as an Alternate Optimization (AO) problem. Classical instances of AO problems include fuzzy regression c-models and fuzzy c-means clustering. Hence, the A-KFD algorithm inherits the convergence properties and characteristics of AO problems.

The set of points for which the A-KFD Algorithm can converge can include certain type of saddle points (i.e., a point that behaves like a local minimizer only when projected along a subset of the variables). However, it is extremely difficult to find examples where convergence occurs to a saddle point rather than to a local minimizer. If the initial estimate is chosen sufficiently near a solution, a local q-linear convergence result is also possible. Further, more detailed convergence can be analyzed in the more general context of regularization networks including SVM type loss functions.

Performance of the A-KFD Algorithm in context of exemplary numerical experiments using various embodiments of the invention is described next. The Algorithm was tested on five publicly available exemplary datasets commonly used in the literature for benchmarking from the University of California, Irvine (UCI) Machine Learning Repository: Ionosphere, Cleveland Heart, Pima Indians, BUPA Liver and Boston Housing.

Additionally, a sixth dataset, a colon CAD dataset, relates to colorectal cancer diagnosis using virtual colonoscopy derived from computer tomographic images. This dataset is referred to as the colon CAD dataset. The dimensionality and size of each dataset are shown in Table 1. The results of experiments over the A-KFD algorithm described above are compared against standard KFD as described in Equation (7) where the kernel model is chosen using a cross-validation tuning procedure. For the choice of family of kernels used in the Algorithm, a family of five kernels is used. A linear kernel (K=AA′) and four Gaussians kernels with μ ε {0.001, 0.01, 0.1, 1}:

$${( {G}_{μ} )}_{i j} = {( K ( A , B ) )}_{i j} = {{ɛ}^{- μ} {‖ {A}_{i}^{′} - {B}_{j} ‖}^{2}}_{i = 1 … , m , j = 1 … , n .} \qquad ( 25 )$$

where A ε R^{m×n}, N=A′ ε R^{n×m}. For all the experiments, in the Algorithm, initial a⁰ was used such that:

$$K = {\sum }_{j = 1}^{k} {a}_{j}^{( i - 1 )} {K}_{j} = A , {A}^{′} + {G}_{1} \qquad ( 26 )$$

That is, the initial kernel is an equally weighted combination of a linear kernel A′A (the kernel with less fitting power) and G₁ (the kernel with the most fitting power). The parameter v required for both methods was chosen to be on the following set {10-3, 10-2, . . . , 100, . . . , 1011, 1012}. To solve the quadratic programming (QP) problem (24) the CPLEX 9.0 QP solver was used, although, since the problem to solve has good properties and it is relatively small in size (k=5 in the experiments here) any publicly available QP solver can be used for this task.

The methodology used in the exemplary experiments is described next:

[1]. Each dataset was normalized between −1 and 1.

[2]. The data set was randomly split into two groups consisting of 70 per cent for training and 30 per cent for testing. The training subset is referred to as “T_{R}” and the training set is referred to as “T_{E}”.

[3]. On the training set T_{R} a ten-fold cross-validation is used for tuning procedure to select “optimal” values for the parameter v in A-KFD and for the parameters v and μ in the standard KFD. The “optimal” values are the parameters values that maximize the ten-fold cross-validation testing correctness. A linear kernel can also be considered as a kernel choice in the standard KFD.

[4]. Using the “optimal” values found in step [3] above a final classification surface (21) is built, and then the performance on the testing set T_{E} is evaluated . Steps [1] to [4] are repeated ten times and the average testing set correctness is reported in Table 1 below:

TABLE 1

| DATA SET | | KFD + KERNEL | |
|---|---|---|---|
| (m × n) | A-KFD | TUNING | P-VALUE |
| IONOSPHERE | 94.7% | 92.73% | 0.03 |
| (351 × 34) | | | |
| HOUSING | 89.9% | 89.4% | 0.40 |
| (506 × 13) | | | |
| HEART | 79.7% | 82.2% | 0.04 |
| (297 × 13) | | | |
| PIMA | 74.1% | 74.4% | 0.7 |
| (768 × 8) | | | |
| BUPA | 70.9% | 70.5% | 0.75 |
| (345 × 6) | | | |

Ten-fold and testing set classification accuracies and p-values for five publicly available datasets (best and statistical significant values in bold).

The average times over the ten runs are reported in Table 2 further below. A paired t-test at 95 per cent confidence level was performed over the ten run results to compare the performance of the two algorithms tested. In most of the experiments, the p-values obtained show that there is no significant difference between A-KFD and the standard KFD where the kernel model is chosen using a cross-validation tuning procedure. Only on two of the datasets, ionosphere and housing, there is a small statistically significant difference for the two methods, with the performance of A-KFD being the better of the two for the ionosphere dataset and the standard tuning being the best for the housing dataset. These results suggest that the two methods are not significantly different regarding generalization accuracy.

In all experiments, the A-KFD algorithm converged in average on 3 or 4 iterations, thus obtaining the final classifier in a considerable faster time than that required for the standard KFD with kernel tuning. Table 2 below shows that A-KFD was up to about 6.3 times faster in one of the cases.

TABLE 2

| DATA SET | | KFD + KERNEL |
|---|---|---|
| (m × n) | A-KFD (SECS.) | TUNING (SECS.) |
| IONOSPHERE | 55.3 | 350.0 |
| (351 × 34) | | |
| HOUSING | 134.4 | 336.9 |
| (506 × 13) | | |
| HEART | 39.7 | 109.2 |
| (297 × 13) | | |
| PIMA | 341.5 | 598.4 |
| (768 × 8) | | |
| BUPA | 48.2 | 81.7 |
| (345 × 6) | | |

Average times in seconds for both methods: AKFD and standard KFD where the kernel width was obtained by tuning.
Times are the averages over ten runs.
Kernel calculation time and ν tuning time are included in both algorithms (Best times are listed in bold).

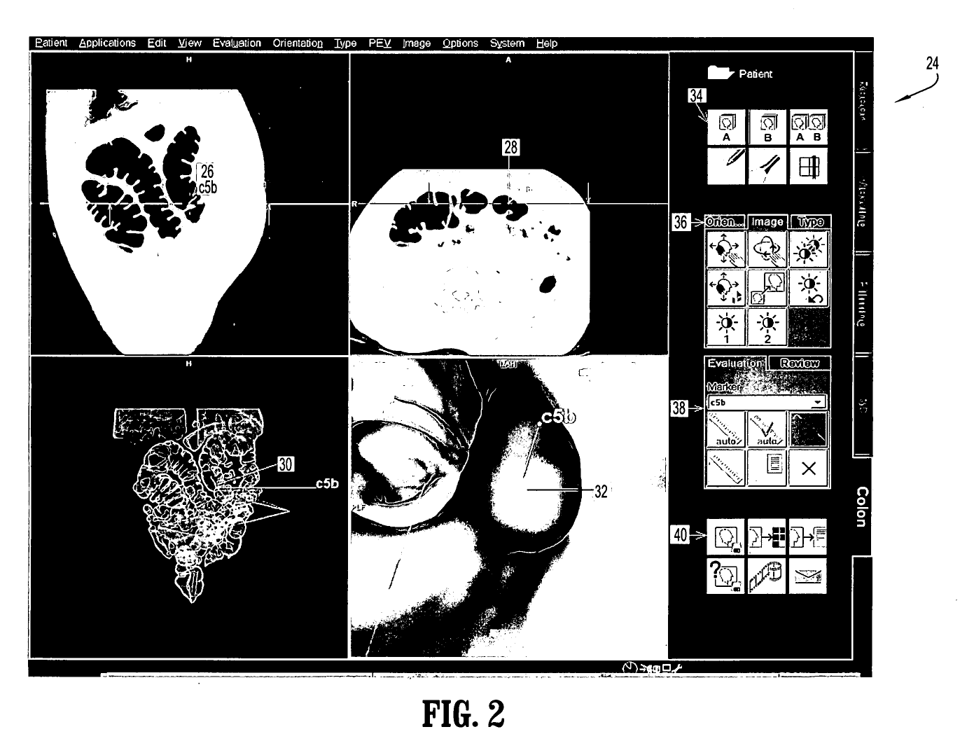

FIG. 2 shows an exemplary colon having a polyp shown in a graphical interface in an exemplary embodiment of the invention. Graphical User Interface (GUI) 24 shows views that a medical expert (e.g., a doctor) reviewing the colon scan will see to detect polyps. The image processing system (not shown) will automatically detect the polyp(s) using A-KFD algorithm in at least one embodiment of the present invention. The detected polyp is displayed for the medical expert for review in the GUI 24. In a first view, the detected polyp 26 is shown (with a marker c5b) as a growth in the colon. Other views show the polyp as 28 and 30. In the last view the detected polyp 32 is shown in a virtual rendering of the colon and the polyp.

The GUI 24 includes image selection functions 34 that can be used to compare multiple images, make notations on the images, etc.; image manipulation functions 36 allow image manipulations such as control of rotation, orientation, brightness, etc.; functional control 38 can be used to detect specific abnormalities; and miscellaneous functions 40 are used to e-mail the scanned image, save the scanned image, etc. Those skilled in the art will appreciate that the GUI controls describe are illustrative and any other type of GUI can be used.

The medical expert can make diagnosis of the polyp and associated problems faster with more accurate and faster automatic detection of polyps. Those skilled in the art will appreciate that colon and polyp are illustrations and any anatomical abnormalities can be detected using the embodiments of the present invention.

Numerical experiments on the Colon CAD dataset are described next. The classification task associated with this dataset is related to colorectal cancer diagnosis. Colorectal cancer is the third most common cancer in both men and women. Recent studies have estimated that in 2003, about 150,000 cases of colon and rectal cancer would be diagnosed in the US, and more than about 57,000 people would die from the disease, accounting for about 10 per cent of all cancer deaths.

A polyp is an small tumor that projects from the inner walls of an intestine or rectum. Early detection of polyps in the colon is critical because polyps can turn into cancerous tumors if they are not detected in the polyp stage. An exemplary database of high-resolution CT images was used in the experiments described next. One hundred and five (105) patient images were selected so as to include positive cases (n=61) as well as negative cases (n=44). The images were preprocessed in order to calculate features based on moments of tissue intensity, volumetric and surface shape and texture characteristics.

The final dataset used in one of the experiments was a balanced subset of the original dataset consisting of 300 candidate structures, where 145 candidates are labeled as polyps and 155 as non-polyps. Each candidate was represented by a vector of 14 features that have the most discriminating power according to a feature selection pre-processing stage. The non-polyp points were chosen from candidates that were consistently misclassified by an existing classifier that was trained to have a very low number of false positives on the entire dataset. Hence, in the given 14 dimensional feature space, the colon CAD dataset is extremely difficult to separate.

For the tests, the same methodology described above for five exemplary datasets was used, which resulted in very similar results as with above exemplary datasets. The standard KFD performed in an average time of 122.0 seconds over ten runs and an average test set correctness of 73.4 per cent. The A-KFD performed in an average time of 41.21 seconds with an average test set correctness of 72.4 per cent. As in the above experiments, a paired t-test at 95 per cent confidence level was performed with a p-value of 0.32>0.05, this indicates that there is no significant difference between both methods in this dataset at the 95 per cent confidence level. Therefore, the A-KFD had the same generalization capabilities and ran almost 3 times faster than the standard KFD.

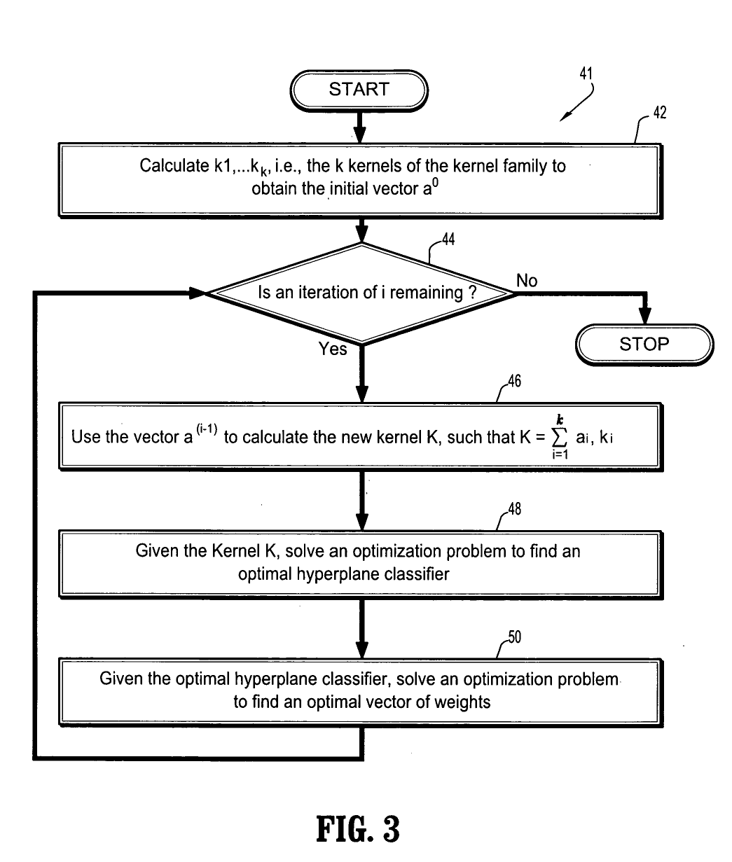

FIG. 3 is a flowchart 41 showing the automatic Kernel Fisher Discriminant (A-KFD) technique that can be used to determine anatomical structures and conditions in a medical image. At a step 42, k kernels of a kernel family (library) are used to generate the kernel K using an initial vector a⁰. A loop runs for a predetermined i number (depending upon the application) of iterations at step 44 that repeats the steps described next. At a step 46, a vector a^{(a-1)} is used to calculate a new kernel K. such that

$${\sum }_{i = 1}^{k} {a}_{i} , {k}_{i} .$$

The kernel K is used to solve a first optimization problem to find an optimal hyperplane classifier at a step 48. At a step 50, a second optimization problem is solved to find optimal vector of weights aⁱ to define a new kernel K as linear combination of kernels in the kernel family.

As discussed above, the optimal weights define a linear combination of kernels from the kernel library that define an optimal kernel or a kernel very suitable for the classification/regression problem at hand, and is used to determine an optimal classifier or regressor. The vector of weights aⁱ⁻¹ is obtained by solving problem (24).

The classifier or regressor thus determined can be used to analyze medical image data. The classifier or regressor can be designed so as to determine any anatomical abnormalities or body conditions.

Various embodiments of the invention can be used to detect anatomical abnormalities or conditions using various medical image scanning techniques. For example candidates can be any of a lung nodule, a polyp, a breast cancer lesion or any anatomical abnormality. Classification and prognosis can be performed for various conditions. For example, lung cancer can be classified from a Lung CAT (Computed Axial Tomography) scan; colon cancer can be classified in a Colon CAT scan; and breast cancer from a X-Ray, a Magnetic Resonance, an Ultra-Sound or a digital mammography scan. Further, prognosis can be performed for lung cancer from a Lung CAT (Computed Axial Tomography) scan; colon cancer from Colon CAT scan; and breast cancer from a X-Ray, a Magnetic Resonance, an Ultra-Sound and a digital mammography scan. Those skilled in the art will appreciate that the above are illustrations of body conditions that can be determined using some exemplary embodiments of the invention, and any other body conditions can also be determined similarly.

A relatively simple procedure for generating heterogeneous Kernel Fisher Discriminant classifier where the kernel model is defined to be a linear combination of members of a potentially larger pre-defined family of heterogeneous kernels is described above. Using this approach, the task of finding an “appropriate” kernel that satisfactorily suits the classification task can be incorporated into the optimization problem to be solved.

In contrast with conventional techniques that also consider linear combination of kernels, the A-KFD requires only: solving a simple nonsingular system of linear equations of the size of the number of training points m and solving a quadratic programming problem that is usually very small since it depends on the predefined number of kernels on the kernel family (five in the exemplary experiments described above). The practical complexity of the A-KFD algorithm does not explicitly depend on the number of kernels on the predefined kernel family.

Empirical results show that the where A-KFD method is several times faster with no significant impact on generalization performance, as compared to the standard KFD where the kernel is selected by a cross-validation tuning procedure. The convergence of the A-KFD algorithm is justified as a special case of the Alternate Optimization (AO) algorithm described.

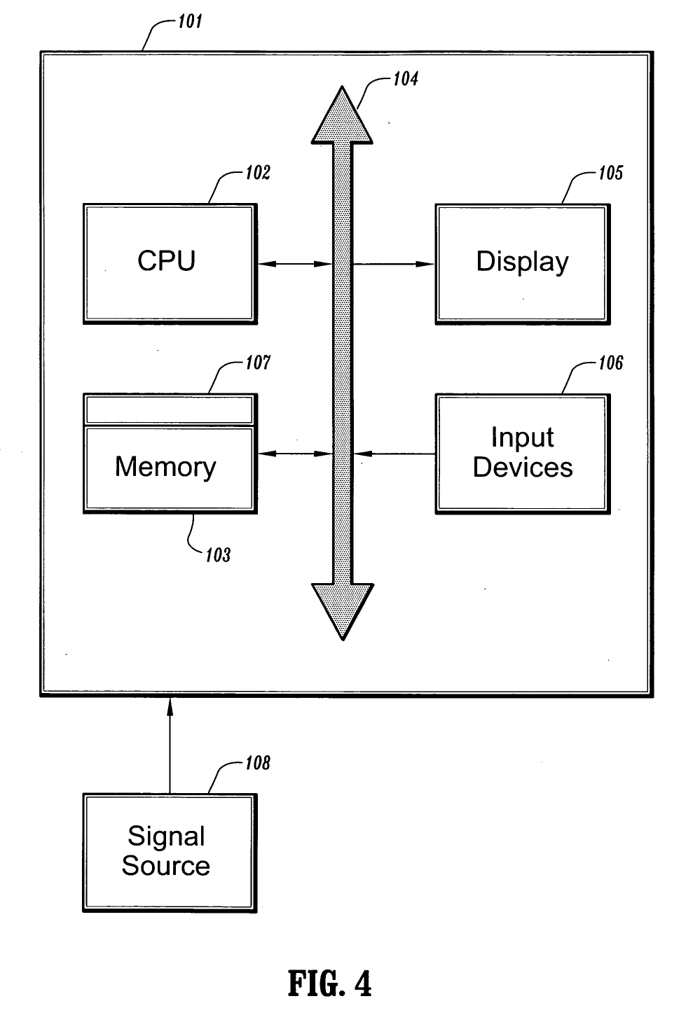

FIG. 4 shows a computer system in an exemplary embodiment of the invention used to implement at least one embodiment of the invention. Referring to FIG. 4, according to an exemplary embodiment of the present invention, a computer system 101 for implementing the invention can comprise, inter alia, a central processing unit (CPU) 102, a memory 103 and an input/output (I/O) interface 104. The computer system 101 is generally coupled through the I/O interface 104 to a display 105 and various input devices 106 such as a mouse and keyboard. The support circuits can include circuits such as cache, power supplies, clock circuits, and a communications bus. The memory 103 can include random access memory (RAM), read only memory (ROM), disk drive, tape drive, etc., or a combination thereof. An exemplary embodiment of the invention can be implemented as a routine 107 that is stored in memory 103 and executed by the CPU 102 to process the signal from the signal source 108. As such, the computer system 101 is a general purpose computer system that becomes a specific purpose computer system when executing the routine 107 of the present invention in an exemplary embodiment of the invention.

The computer platform 101 also includes an operating system and micro instruction code. The various processes and functions described herein may either be part of the micro instruction code or part of the application program (or a combination thereof) which is executed via the operating system. In addition, various other peripheral devices may be connected to the computer platform such as an additional data storage device and a printing device.

It is to be further understood that, because some of the constituent system components and method steps depicted in the accompanying figures may be implemented in software, the actual connections between the system components (or the process steps) may differ depending upon the manner in which the present invention is programmed in an exemplary embodiment of the invention. Given the teachings of the present invention provided herein, one of ordinary skill in the related art will be able to contemplate these and similar implementations or configurations of the present invention.

While the present invention has been particularly shown and described with reference to exemplary embodiments thereof, it will be understood by those skilled in the art that various changes in form and details may be made therein without departing from the spirit and scope of the present invention as defined by the appended claims.

1. A method for analyzing an image data-space to locate one or more candidates, the method comprising the steps of:
creating a library of one or more kernels, wherein each kernel from the library of the kernels maps the image data-space to a first data-space using at least one mapping function;
learning a linear combination of the kernels in an automatic manner to generate at least one of a classifier and a regressor, wherein the linear combination comprises at least two kernels from the library of kernels;
applying the linear combination of kernels by using at least one of the classifier and the regressor to the first data-space to generate a classified image-data space; and
detecting the presence or absence of at least one of the candidates in the classified image-data space.

2. The method of claim 1, wherein the step of learning the kernels in an automatic manner further comprises the step of:
determining one or more optimal weights in the linear combination of the kernels.

3. The method of claim 2, wherein the linear combination and the optimal weights are determined using an equation:
$$K = {\sum }_{j = 1}^{k} {a}_{j}^{( i - 1 )} {K}_{j} .$$

4. The method of claim 1, wherein the kernels are represented by an equation: K(A, A′): R^{m×n}×R^{n×m} [symbol] R^{m×m}.

5. The method of claim 4, wherein at least one of the kernels is a Gaussian kernel represented by an equation:
K(A,A′)ᵢⱼ=ε^{-μ∥A}^{ᵢ}^{-A}^{ⱼ}^{∥}^{₂}^{²}, i,j=1, . . . ,m.

6. The method of claim 4, wherein at least one of the kernels is a polynomial kernel represented by an equation:
K(A, A′)ᵢⱼ=(A′ᵢAⱼ)^{k}+b, i,j=1, . . . m.

7. The method of claim 1, wherein at least one of the classifier and the regressor is represented by the equation:
$${\sum }_{j = 1}^{k} ( {a}_{j} {K}_{j} ( A , {A}^{′} ) ) υ - e γ .$$

8. The method of claim 1, wherein the step of learning further comprises the step of:
solving an optimization problem represented by an equation:
$${min}_{( υ , γ , a \geq 0 ) ∈ {R}^{m + 1}} v \frac{1}{2} ‖ d - {( ( {\sum }_{j = 1}^{k} ( {a}_{j} {K}_{j} ( A , {A}^{′} ) ) υ - e γ ) ‖}^{2} + \frac{1}{2} ( {υ}^{′} υ ) + \frac{1}{2} {a}^{′} a .$$

9. The method of claim 8, wherein the optimization problem is solved using an iteration based on an Alternate Optimization (AO), wherein the Alternate Optimization comprises one or more convex problems.

10. The method of claim 1, wherein the optimization problem is solved using an Expectation Maximization (EM) procedure.

11. The method of claim 1, wherein the at least one of the candidates is a lung nodule, a polyp, a breast cancer lesion and an anatomical abnormality.

12. The method of claim 1, further comprising the step of:
classifying at least one of a lung cancer when the image data-space is a Lung CAT (Computed Axial Tomography) scan, a colon cancer when the image data-space is a Colon CAT scan, and a breast cancer when the image data-space is at least one of a X-Ray, a Magnetic Resonance, an Ultra-Sound and a digital mammography scan.

13. The method of claim 1, further comprising the step of:
performing prognosis for at least one of a lung cancer when the image data-space is a Lung CAT (Computed Axial Tomography) scan, a colon cancer when the image data-space is a Colon CAT scan, and a breast cancer when the image data-space is at least one of a X-Ray, a Magnetic Resonance, an Ultra-Sound and a digital mammography scan.

14. The method of claim 1, wherein the step of learning uses at least one of a support vector machines technique, a least-square support vector machines technique and a Kernel Fisher Discriminant technique.

15. The method of claim 1, wherein the step of learning uses one or more weak kernels from the library of kernels for automatic feature selection in the image data-space, wherein the weak kernels depend on only one input feature.

16. The method of claim 15, wherein the weak kernels comprise weak column kernels that depend on a subset of the centers of the kernels in the library.

17. A method for finding a regularized network that solves a nonlinear classification problem, the method comprising the steps of:
creating a library of kernels, wherein each kernel from the library of the kernels maps an input data-space to a first data-space using at least one mapping function;
determining a linear combination of the kernels;
solving a first convex Quadratic Programming (QP) problem using the linear combination of kernels to generate a hyperplane;
solving a second convex QP problem using the solved first QP and the hyperplane to determine at least one of a classifier and a regressor; and
generate a classified data space by applying at least one of the classifier and a regressor to the first data-space.

18. The method of claim 17, wherein the step of creating further comprises the step of:
calculating K1, . . . ,Kₖ, the k kernels of the kernel family, where for each i, Kᵢ=Kᵢ(A,A′).

19. The method of claim 18, wherein the step of determining further comprises the step of:
calculating
$$K = {\sum }_{j = 1}^{k} {a}_{j}^{( i - 1 )} {K}_{j} ,$$
for each given a⁽ⁱ⁻¹⁾.

20. The method of claim 19, wherein the step of solving the first convex QP further comprises the step of:
solving
$${min}_{( S ) ∈ ( {R}^{m + 1} )} F ( S , \hat{a} ) = {min}_{( υ , γ ) ∈ {R}^{m + 1}} v \frac{1}{2} {‖ d - ( \hat{K} υ - e γ ) ‖}^{2} + \frac{1}{2} ( {υ}^{′} υ + {γ}^{2} )$$
to obtain (v⁽ⁱ⁾,γ⁽ⁱ⁾).

21. The method of claim 20, wherein the step of solving the second convex QP further comprises the step of:
solving
$$\begin{matrix}{min}_{T \geq 0 ∈ ( {R}^{k} )} F ( \hat{S} , T ) = {min}_{a \geq 0 ∈ ( {R}^{k} )} F ( \hat{S} , a ) \\ = {min}_{a \geq 0 ∈ {R}^{k}} v \frac{1}{2} {‖ d - ( ( {\sum }_{j = 1}^{k} {Λ}_{j} {a}_{j} ) - e \hat{γ} ) ‖}^{2} + \frac{1}{2} ( {a}^{′} a )\end{matrix}$$
to obtain aⁱ.

22. The method of claim 17 further comprising the step of:
iterating to perform the steps of determining the linear combination, solving the first convex QP and the second convex QP for a predetermined iteration threshold times.

23. A program storage device readable by machine, tangibly embodying a program of instructions executable by the machine to perform method steps for analyzing image data-space to locate one or more candidates, the method steps comprising:
creating a library of one or more kernels, wherein each kernel from the library of the kernels maps the image data-space to a first data-space using at least one mapping function;
learning a linear combination of kernels in an automatic manner to generate at least one of a classifier and a regressor, wherein the linear combination comprises at least two kernels from the library of kernels;
applying the linear combination of kernels by using at least one of the classifier and the regressor to the first data-space to generate a classified image-data space; and
detecting the presence or absence of at least one of the candidates in the classified image-data space.

24. The device of claim 23, wherein the instructions for the step of learning further comprises the step of:
determining one or more optimal weights in the linear combination of the kernels.

25. The device of claim 24, wherein the linear combination and the optimal weights are determined using an equation:
$$K = {\sum }_{j = 1}^{k} {a}_{j}^{( i - 1 )} {K}_{j} .$$

26. The device of claim 23, wherein at least one of the classifier and the regressor is represented by the equation:
$${\sum }_{j = 1}^{k} ( {a}_{j} {K}_{j} ( A , {A}^{′} ) ) υ - e γ .$$

27. The device of claim 23, wherein the instructions for the step of learning are performed by solving an optimization problem represented by an equation:
$${min}_{( υ , γ , a \geq 0 ) ∈ {R}^{m + 1}} v \frac{1}{2} ‖ d - {( ( {\sum }_{j = 1}^{k} ( {a}_{j} {K}_{j} ( A , {A}^{′} ) ) υ - e γ ) ‖}^{2} + \frac{1}{2} ( {υ}^{′} υ ) + \frac{1}{2} {a}^{′} a .$$

28. The device of claim 23, wherein the at least one of the candidates is a lung nodule, a polyp, a breast cancer lesion and an anatomical abnormality.

29. The device of claim 23, wherein the instructions further comprising the step of:
classifying at least one of lung cancer when the image data-space is a Lung CAT (Computed Axial Tomography) scan, a colon cancer when the image data-space is a Colon CAT scan, and breast cancer when the image data-space is at least one of a X-Ray, a Magnetic Resonance, an Ultra-Sound and a digital mammography scan.

30. The device of claim 23, wherein the instructions further comprising the step of:
performing prognosis for at least one of lung cancer when the image data-space is a Lung CAT (Computed Axial Tomography) scan, a colon cancer when the image data-space is a Colon CAT scan, and a breast cancer when the image data-space is at least one of a X-Ray, a Magnetic Resonance, an Ultra-Sound and digital mammography scan.
